SEQUENCE LISTING (11 sequences)

SEQ ID NO: 1 - protein, 6 aa, synthetic construct
REESGE

SEQ ID NO: 2 - protein, 7 aa, synthetic construct
REESGEP

SEQ ID NO: 3 - protein, 9 aa, synthetic construct
KEEDEESGE

SEQ ID NO: 4 - protein, 8 aa, synthetic construct
KPREESGE

SEQ ID NO: 5 - protein, 8 aa, synthetic construct
LDEESGEP

SEQ ID NO: 6 - protein, 9 aa, synthetic construct
REESDKPMY

SEQ ID NO: 7 - protein, 8 aa, synthetic construct
PREESDKP

SEQ ID NO: 8 - protein, 7 aa, synthetic construct
REESGEL

SEQ ID NO: 9 - skipped

SEQ ID NO: 10 - protein, 9 aa, synthetic construct
  VARIANT 1: note = Absent or present in repeats of up to 10; each X, if present, is independently selected from any amino acid
  VARIANT 2: note = Arginine (R) or Aspartic acid (D)
  VARIANT 9: note = Absent or present in repeats of up to 9; each X, if present, is independently selected from any amino acid
XXEESGEPX

SEQ ID NO: 11 - protein, 8 aa, synthetic construct
  VARIANT 1: note = Leucine (L) or absent
  VARIANT 2: note = Arginine (R) or Aspartic acid (D)
XXEESGEP

(19) United States
(12) Patent Application Publication
(10) Pub. No.: US 2025/0127845 A1
(43) Pub. Date: Apr. 24, 2025

(54) MEDICINAL USES OF OLIGOPEPTIDES IN COMBINATION WITH AN ANTIANDROGEN

(71) Applicant: HBC IMMUNOLOGY INC., Menlo Park, CA (US)

(72) Inventor: Bomi FRAMROZE, Portola Valley, CA (US)

(73) Assignee: HBC Immunology Inc., Menlo Park, CA (US)

(21) Appl. No.: 18/832,867

(22) PCT Filed: Feb. 3, 2023
(86) PCT No.: PCT/US2023/061992

§ 371 Date: Jul. 24, 2024

Related U.S. Application Data
(60) Provisional application No. 63/306,979, filed on Feb. 4, 2022.

(51) Int. Cl. A61K 38/08; A61K 9/00; A61K 31/277; A61K 31/4166; A61K 38/01; A61K 45/06; A61P 35/00

(52) U.S. Cl. CPC A61K 38/08; A61K 9/0053; A61K 31/277; A61K 31/4166; A61K 38/012; A61K 45/06; A61P 35/00

(57) ABSTRACT

The present disclosure relates to methods and medicaments for inhibiting proliferation of a carcinoma cell by contacting the carcinoma cell with an antiandrogen in combination with a formulation comprising an oligopeptide capable of increasing expression of ferritin heavy chain 1 (FTH1) by epithelial cells. The methods and medicaments are suitable for treating prostate cancer,

### CROSS-REFERENCE TO RELATED APPLICATIONS

This application claims priority to and the benefit of U.S. Provisional Application No. 63/306,979, filed Feb. 4, 2022, the content of which is incorporated herein by reference in its entirety.

### REFERENCE TO AN ELECTRONIC SEQUENCE LISTING

The content of the electronic sequence listing (197732000840SEQLIST.xml; Size: 12,028 bytes; and Date of Creation: Feb. 2, 2023) is herein incorporated by reference in its entirety.

### FIELD

The present disclosure relates to methods and medicaments for inhibiting proliferation of a carcinoma cell by contacting the carcinoma cell with an antiandrogen in combination with a formulation comprising an oligopeptide capable of increasing expression of ferritin heavy chain 1 (FTH1) by epithelial cells. The methods and medicaments are suitable for treating prostate cancer.

### BACKGROUND

Prostate cancer (PC) is one of the most common male-specific cancers worldwide and is the third leading cause of death. Current treatment options for PC are prostatectomy, hormonal therapy, chemotherapy or radiotherapy with low success rates, and tumors developing resistance and cancer relapsing soon after treatment. About 10% of men treated with curative intent will develop metastasis over time. Current therapy for metastatic disease is based on androgen deprivation and/or androgen receptor (AR) inhibition such as treatment with bicalutamide. Unfortunately, virtually all patients exposed to longer-term treatment become refractory to antiandrogen therapy.

Thus, there is a persistent unmet need for more effective therapies for treating locally advanced and metastatic prostate cancer. In addition, combination therapies for enhancing the effect of antiandrogens are desirable.

### BRIEF SUMMARY

The present disclosure relates to methods and medicaments for inhibiting proliferation of a carcinoma cell by contacting the carcinoma cell with an antiandrogen in combination with a formulation comprising an oligopeptide capable of increasing expression of ferritin heavy chain 1 (FTH1) by epithelial cells. The methods and medicaments are suitable for treating prostate cancer.

### BRIEF DESCRIPTION OF THE DRAWINGS

FIG. 1 is a plot of clonogenic assay results showing the effect of treatment of LNCaP cells (an androgen receptor-positive cell line) with salmon protein hydrolysate (SPH) alone or in combination with an antiandrogen (bicalutamine=BIC).

FIG. 2 is a plot of clonogenic assay results showing the effect of treatment of PC3 cells (an androgen receptor-negative cell line) with salmon protein hydrolysate (SPH) alone or in combination with an antiandrogen (bicalutamine=BIC).

FIG. 3 is an alignment of the amino acid sequence of the eight major peptides from the three bioactive fractions FRP18, FRP20 and FRP30, along with a shared “EES” motif, a consensus sequence and sequence identifiers.

FIG. 4 is a plot of clonogenic assay results showing the effect of treatment of VCaP cells (an androgen receptor-positive cell line) with salmon protein hydrolysate (SPH) alone or in combination with an antiandrogen (enzalutamide=ENZ).

FIG. 5 is a plot of clonogenic assay results showing the effect of treatment of VCaP-EnzR cells (an androgen receptor-positive cell line that is relatively resistant to treatment with the antiandrogen, enzalutamide) with salmon protein hydrolysate (SPH) alone or in combination with an antiandrogen (enzalutamide=ENZ).

FIG. 6 is a plot of clonogenic assay results showing the effect of treatment of LNCaP cells (an androgen receptor-positive cell line) with an antiandrogen (bicalutamine=BIC) alone or in combination with either SPH or one of eight oligopeptides.

FIG. 7 is a plot of clonogenic assay results showing the effect of treatment of PC3 cells (an androgen receptor-negative cell line) with an antiandrogen (bicalutamine=BIC) alone or in combination with either SPH or one of eight oligopeptides.

FIG. 8 is a plot of clonogenic assay results showing the effect of treatment of VCaP cells (an androgen receptor-positive cell line) with an antiandrogen (enzalutamide=ENZ) alone or in combination with either SPH or one of eight oligopeptides.

FIG. 9 is a plot of clonogenic assay results showing the effect of treatment of VCaP-EnzR cells (an androgen receptor-positive cell line that is relatively resistant to treatment with the antiandrogen, enzalutamide) with an antiandrogen (enzalutamide=ENZ) alone or in combination with either SPH or one of eight oligopeptides.

### DETAILED DESCRIPTION

The present disclosure relates to methods and medicaments for inhibiting proliferation of a carcinoma cell by contacting the carcinoma cell with an antiandrogen in combination with a formulation comprising an oligopeptide capable of increasing expression of ferritin heavy chain 1 (FTH1) by epithelial cells. The methods and medicaments are suitable for treating prostate cancer.

Iron homeostasis has been shown to be an important modulator of prostate cancer (PC) onset and progression. Cellular iron redox is maintained by a balance between the storage protein ferritin, iron transporters and the exporter ferroportin. For instance, direct iron supplementation has recently been shown to be toxic for prostate cancer cells in cellular and murine models (Bordini et al., Clin Cancer Res, 26:6387-6398, 2020). Additionally, iron supplementation in combination with the androgen inhibitor bicalutamide inhibited growth of androgen receptor (AR) negative (−ve) PC cell lines than either treatment alone. However, directly administering large doses of iron is contemplated to be clinically infeasible due to multiple side-effects.

Additionally, the ferritin heavy chain 1 (FTH1) transcript and multiple FTH1pseudogenes have been identified as targets of oncogenic microRNAs. Increasing FTH1expression by impairing the competing endogenous RNA crosstalk resulted in a decrease in oncogenesis in vitro and in vivo (Chan et al., Nucleic Acids Research, 46(4):1998-2011, 2018). Moreover, both AR+ve (LNCaP, VCaP) and AR−ve (PC3, DU145) prostate cancer cells have been found to over-express transferrin receptor 1 (TFR1) and under-express FTH1 proteins relative to normal prostate epithelial cells (Keer et al., J Urol, 143(2):381-385, 1990; and Chan et al., supra, 2018). Other reports have described the role of iron homeostasis dysfunction and tumor-growth via the p53-ISCU pathway (Funaguchi et al., Sci Rep, 5:16497, 2015) and via the hepcidin pathway (Tesfay et al., Cancer Res, 75(11):2254-2263, 2015), both of which control iron homeostasis and which were found to be significantly upregulated in PC cells.

Previously, prostate cancer cells were found to retain higher levels of metabolically active iron, known as the labile iron pool, than benign prostate epithelial cells (Funaguchi et al., Sci Rep, 5:16497, 2015). Two proteins important for modulation of intracellular iron levels are the iron import protein TFR1, and the iron storage protein ferritin, which is a globular protein complex made up of FTH1 and ferritin light chain (FTL) subunits.

Levels of TFR1 and FTH1 proteins were measured by analysis of Western blots of lysates from normal prostate epithelial cells (PrEC) and two prostate cancer cell lines (LNCaP, an AR+ve cell line; and PC3, an AR−ve cell line). Expression of the TFR1 protein was markedly increased in both prostate cancer cell lines as compared to normal prostate epithelial cells. In contrast, FTH1 protein levels were decreased to barely detectable levels in both prostate cancer cells, while being faintly expressed in PrEC cells. Elevated TFR1 and depressed FTH1 expression are associated with a high iron uptake and a low iron storage capability.

As described herein in Example 2, salmon protein hydrolysate (SPH) has now been found to inhibit proliferation of AR+ve and AR−ve prostate cancer cells when cultured in vitro in the presence of bicalutamide. Bicalutamide is an AR antagonist type of nonsteroidal antiandrogen, which is used to treat metastatic and locally advanced prostate cancer. Increasing the effect of bicalutamide and other antiandrogens in vivo by use in combination with an oligopeptide therapeutic agent such as SPH or bioactive peptide(s) of SPH is contemplated to improve its utility in treating prostate cancer, and other indications in which bicalutamide is prescribed off-label.

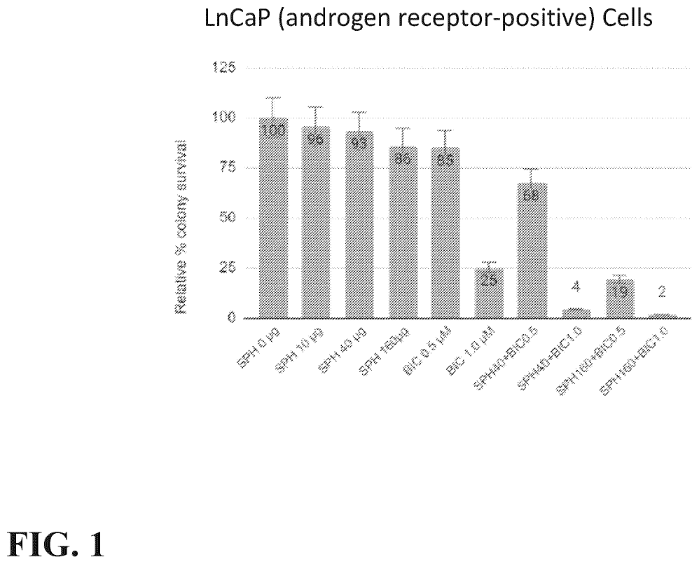

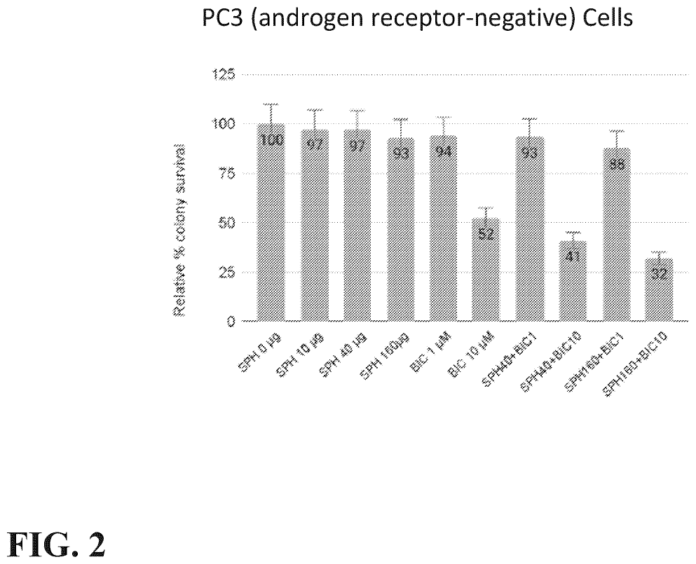

Specifically, as shown in FIG. 1 and FIG. 2, and described in Example 2, SPH as a mono-treatment had no effect on LNCaP (AR+ve) and PC3 (AR−ve) cell proliferation. However, co-treatment of SPH at both low (40 μg/ml) and high (160 μg/ml) concentration together with a minimal inhibitory concentration (MIC) of bicalutamide (BIC) of 1 μM for LNCaP and 10 μM for PC3 significantly increased the therapeutic effect of bicalutamide treatment, in both cell lines. In LNCaP AR+ve cells, SPH co-treatment (160 μg/ml) decreased the relative percent of cell survival of 0.5 μM BIC treatment from 85% to 19%, and decreased the relative percent of cell survival of 1.0 μM BIC treatment from 25% to 2%. Likewise, in PC3 AR−ve cells, SPH co-treatment (160 μg/ml) decreased the relative percent of cell survival of 10 μM BIC treatment from 52% to 32%.

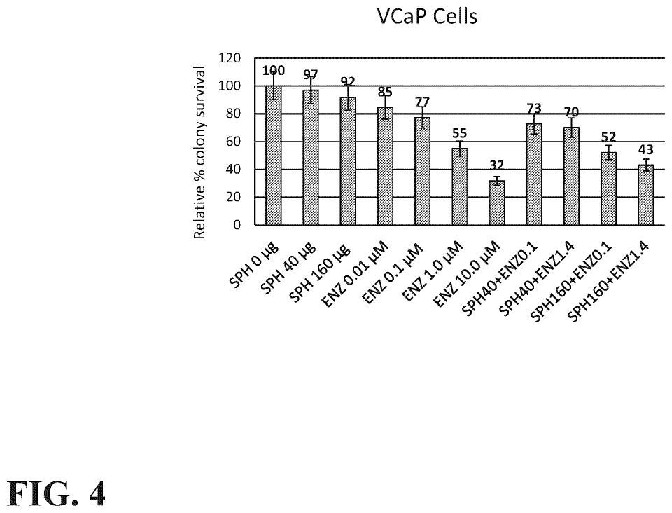

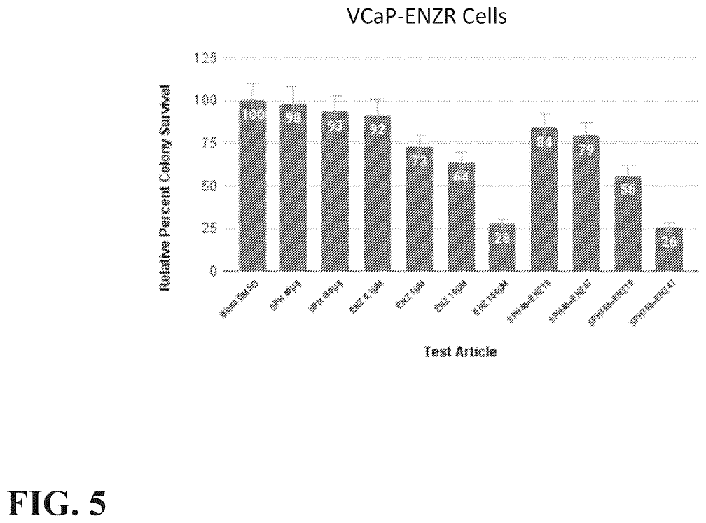

Additionally, as shown in FIG. 4 and FIG. 5, and described in Example 2, SPH as a mono-treatment had little effect on VCaP (AR+ve) and VCaP-ENZR (AR+ve) cell proliferation. However, co-treatment of SPH at high (160 μg/ml) concentration together with a less than minimal inhibitory concentration (MIC) of enzalutamide (ENZ) increased the therapeutic effect of enzalutamide treatment, in both cell lines. In VCaP cells, SPH co-treatment (160 μg/ml SPH) decreased the relative percent of cell survival of 0.1 μM ENZ treatment from 77% to 52%. Similarly, in VCaP-ENZR cells, SPH co-treatment (160 μg/ml SPH) decreased the relative percent of cell survival of 10 μM ENZ treatment from 64% to 56%.

Moreover, gene expression was attenuated in both LNCaP and PC3 prostate cancer cell lines when treated with SPH and bicalutamide (BIC). Likewise, gene expression was attenuated in both VCaP and VCaP-ENZR prostate cancer cell lines when treated with SPH and enzalutamide (ENZ). In particular, FTH1 was up-regulated and TFRC was down-regulated by greater than 2-fold relative to the housekeeping gene ACTB in prostate cancer cells cultured in the presence of both SPH and BIC or ENZ, but not BIC or ENZ alone (see, Table 2-1 and Table 2-2).

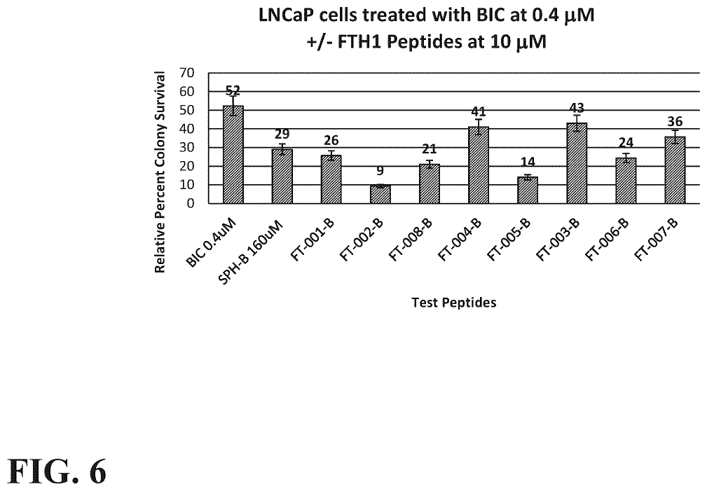

The desirable properties of SPH treatment in combination with BIC or ENZ were also found upon treatment of prostate cancer cells with BIC or ENZ in combination with synthetic oligopeptides with amino acid sequences identified from active fractions of SPH. The most potent anti-proliferative effects were observed when prostate cancer cells were treated with oligopeptide FT-002 or FT-005 in combination with BIC or ENZ (see, Example 3 and FIGS. 6-9). Also, FTH1 was up-regulated and TFRC was down-regulated by to the same or greater extent in prostate cancer cells cultured in the presence of an oligopeptide in combination with BIC or ENZ than was observed when prostate cancer cells were cultured in the presence of SPH and BIC or ENZ (see, Table 3-1 and Table 3-2).

### Definitions

As used herein and in the appended claims, the singular forms “a,” “or,” and “the” include plural references unless the context indicates otherwise. For example, “an excipient” includes one or more excipients.

It is understood that aspects and embodiments described herein as “comprising” include “consisting of” and/or “consisting essentially of” aspects and embodiments.

The term “about” as used herein in reference to a value describes from 90% to 110% of that value. For instance, about a 2-fold change in FTH1 mRNA expression includes a change of 1.8-fold to 2.2-fold, and includes 2.0-fold change in FTH1 mRNA.

As used herein, the term “androgen receptor” refers to a receptor that is activated by binding testosterone or dihydrotestosterone (androgenic hormones). The androgen receptor is also known as the “nuclear receptor subfamily 3, group C, member 4” and “NR3C4.” The amino acid sequence of the human androgen receptor is set forth as GenBank Accession No. NP_000035 (isoform 1), and the nucleic acid sequence of the human androgen receptor is set forth as GenBank Accession No. NM_000044 (variant 1).

As used herein, the terms “ferritin heavy chain 1” and “FTH1” refer to the nucleic acid sequence encoding the “ferritin heavy chain” protein, which is also known as the “ferritin H subunit.” The amino acid sequence of the human ferritin heavy chain is set forth as GenBank Accession No. NP_002023, and the mRNA sequence is set forth as GenBank Accession No. NM_002032, with the coding sequence extending from nucleotides 210-761.

The term “isolated” as used herein in reference to molecules (e.g., oligopeptides), refers to molecules that are removed or otherwise purified from their natural or synthetic environment. Substantially “isolated” molecules are at least 75% free, preferably at least 90% free, more preferably at least 95%, 96%, 97%, 98% or 99% free from other components. For instance, an “isolated oligopeptide consisting of the amino acid sequence of SEQ ID NO:9” is at least at least 75% free of peptides and proteins that do not comprise the amino acid sequence of SEQ ID NO:9.

The term “increasing” and grammatical equivalents as used herein in reference to expression or levels of FTH1 mRNA, refers to causing FTH1 mRNA to become greater in amount. Preferably, an increase in FTH1 mRNA encompasses a statistically significant increase. Preferably, an increase in FTH1 mRNA encompasses an increase of about 2 to about 200 fold, about 2 to 20 fold, or about 2 to 4 fold, or more preferably at least 2.0, 2.1, 2.2, 2.3, 2.4, 2.5, 3.0, 3.1, 3.2, 3.3, 3.4 or 3.5 of a baseline FTH1 mRNA level.

The term “decreasing” and grammatical equivalents as used herein in reference to expression or levels of TFRC mRNA, refers to causing TFRC mRNA to become less in amount. Preferably, a decrease in TFRC mRNA encompasses a statistically significant decrease. Preferably, a decrease in TFRC mRNA encompasses a decrease of about 2 to about 200 fold, about 2 to 20 fold, or about 2 to 4 fold, or more preferably at most 0.8, 0.8, 0.6, 0.5, 0.4, 0.3, 0.2 or 0.1 of a baseline TFRC mRNA level.

As used herein, the terms “treating” and “treatment” refer to an approach for obtaining beneficial or desired results, including clinical results. Beneficial or desired clinical results include, but are not limited to, alleviation or amelioration of one or more symptoms, diminishment of extent of disease, stabilized (i.e., not worsening) state of disease, preventing spread of disease, delay or slowing of disease progression, amelioration or palliation of the disease state, and remission (whether partial or total), whether detectable or undetectable. “Treatment” can also mean prolonging survival as compared to expected survival if not receiving treatment. As such, the terms “treating” and “treatment” as used herein, do not require complete alleviation of signs or symptoms, do not require a cure, and specifically include protocols that have a modest effect on the individual

An “effective amount” of an agent disclosed herein (e.g., isolated oligopeptide or formulation thereof) is an amount sufficient to carry out a specifically stated purpose. An “effective amount” may be determined empirically in relation to the stated purpose. An “effective amount” or an “amount sufficient” of an agent is that amount adequate to affect a desired biological effect, such as a beneficial result, including a beneficial clinical result. The term “therapeutically effective amount” refers to an amount of an agent (e.g., isolated oligopeptide or formulation thereof) effective to “treat” a disease or disorder in a subject (e.g., a mammal such as a human). An “effective amount” or an “amount sufficient” of an agent may be administered in one or more doses.

Administration “in combination with” or “in addition to” one or more further therapeutic agents includes simultaneous (concurrent) and consecutive administration in any order.

The terms “individual” and “subject” refer to mammals. “Mammals” include, but are not limited to, humans, non-human primates (e.g., monkeys), farm animals, sport animals, rodents (e.g., mice and rats) and pets (e.g., dogs and cats).

### I. Salmon Protein Hydrolysate and Isolated Oligopeptides

Salmon protein hydrolysate (SPH) is a complex blend of over 500 peptides obtained by enzymatic hydrolysis of salmon protein material. When properly prepared, as in ProGo™ (manufactured by Hofseth BioCare ASA, Alesund, Norway), SPH contains great than 97% protein content (w/w), primarily made up of oligopeptides and peptides with a molecular weight of less than about 1000-3000 daltons. SPH such as ProGo™, or other formulation prepared as described (see, e.g., Example 1 herein; and Examples 1, 4 and 5 of US 2021/0252099), has previously been shown to be “bioactive” in that it regulates expression of oxidative stress-related genes. In particular, SPH was shown to upregulate expression of FTH1 mRNA and heme oxygenase 1 (HMOX1) mRNA, and down-regulate arachidonate 12-lipoxygenase (ALOX12) mRNA in normal epithelial cells (see, e.g., Example 8 of US 2021/0252099).

Salmon protein hydrolysates and other fish protein hydrolysates, however, are very complex compositions containing hundreds of peptides, many of which may be benign inactive peptides, while others may have undesirable activities. Thus, in some embodiments, an isolated oligopeptide possessing the desired bioactivity of SPH (e.g., ability to cause an increase in FTH1 mRNA expression) is preferable to SPH for use in the methods and medicaments of the present disclosure.

The isolated oligopeptides of the present disclosure consist of the amino acid sequence of Xm(R/D)EES(G/D)(E/K)Xn (SEQ ID NO:9), in which m and n are integers independently selected from the range of from 0-10, and each X, if present, is independently selected from any amino acid. Specifically, in some embodiments, numbers of m and n are each selected from 0, 1, 2, 3, 4, 5, 6, 7, 8, 9 or 10. Thus, the claimed oligopeptides are from 6 to 26 residues in length. In some embodiments, the oligopeptide is no less than 6, 7, 8, 9, 10, 11, 12, 13, 14, 15, 16, 17, 18, 19, 20, 21, 22, 23, 24 or 25 residues in length, and/or the oligopeptide is no more than 26, 25, 24, 23, 22, 21, 20, 19, 18, 17, 16, 15, 14, 13, 12, 11, 10, 9, 8, or 7 residues in length, in which the lower limit is less than the upper limit. In some embodiments, the isolated oligopeptide comprises the amino acid sequence of REESGE (SEQ ID NO:1). In some embodiments, the isolated oligopeptide comprises the amino acid sequence of REESGEP (SEQ ID NO: 2). In some embodiments, the isolated oligopeptide comprises the amino acid sequence of KEEDEESGE (SEQ ID NO:3). In some embodiments the isolated oligopeptide comprises the amino acid sequence of KPREESGE (SEQ ID NO:4). In some embodiments the isolated oligopeptide comprises the amino acid sequence of LDEESGEP (SEQ ID NO:5). In some embodiments, the isolated oligopeptide comprises the amino acid sequence of REESDKPMY (SEQ ID NO:6). In some embodiments, the isolated oligopeptide comprises the amino acid sequence of PREESDKP (SEQ ID NO:7). In some embodiments, the isolated oligopeptide comprises the amino acid sequence of REESGEL (SEQ ID NO:8). In some embodiments, the isolated oligopeptide comprises an amino acid sequence having at least 90% (e.g., at least 91%, 92%, 93%, 94%, 95%, 96%, 97%, 98%, 99%, or 100%) amino acid sequence identity to any one of SEQ ID NOS: 1-8.

In some embodiments, the isolated oligopeptides of the present disclosure consist of the amino acid sequence of Xp(R/D)EESGEPXq (Consensus No. 2/SEQ ID NO:10), in which p is an integer selected from the range of from 0-10, q is an integer selected from the range of from 0-9, and each X, if present, is independently selected from any amino acid. Thus, in some embodiments, the claimed oligopeptides are from 7 to 26 residues in length. In some embodiments, the oligopeptide is no less than 7, 8, 9, 10, 11, 12, 13, 14, 15, 16, 17, 18, 19, 20, 21, 22, 23, 24 or 25 residues in length, and/or the oligopeptide is no more than 26, 25, 24, 23, 22, 21, 20, 19, 18, 17, 16, 15, 14, 13, 12, 11, 10, 9, or 8 residues in length, in which the lower limit is less than the upper limit. In some embodiments, the oligopeptides comprise the amino acid sequence of Xr(R/D)EESGEP (Consensus No. 3/SEQ ID NO:11), in which Xr is leucine or absent. In some embodiments, the isolated oligopeptide comprises the amino acid sequence of REESGEP (SEQ ID NO:2). In some embodiments the isolated oligopeptide comprises the amino acid sequence of LDEESGEP (SEQ ID NO:5).

In preferred embodiments, the isolated oligopeptides are capable of increasing expression of ferritin heavy chain 1 (FTH1) mRNA by carcinoma cells contacted with the oligopeptides in the presence of an antiandrogen. The increase in expression of FTH1 mRNA as a result of contact with the oligopeptide is relative to carcinoma cells contacted with the androgen in the absence of the oligopeptide, relative to carcinoma cells cultured under the same condition except for the absence of the oligopeptide, or except for the presence of a negative control oligopeptide (e.g., oligopeptide about the same length, but which does not comprise SEQ ID NO: 9). In some embodiments, the carcinoma cells are mammalian cells. In some preferred embodiments, the mammalian cells are human cells. In an exemplary embodiment, the carcinoma cells are LNCaP cells. In another exemplary embodiment, the carcinoma cells are PC3 cells.

In preferred embodiments, the isolated oligopeptides are capable of decreasing expression of transferrin receptor 1 (TFRC) mRNA by carcinoma cells contacted with the oligopeptides in the presence of an antiandrogen. The decrease in expression of TFRC mRNA as a result of contact with the oligopeptide is relative to carcinoma cells contacted with the androgen in the absence of the oligopeptide, relative to carcinoma cells cultured under the same condition except for the absence of the oligopeptide, or except for the presence of a negative control oligopeptide (e.g., oligopeptide about the same length, but which does not comprise SEQ ID NO: 9). In some embodiments, the carcinoma cells are mammalian cells. In some preferred embodiments, the mammalian cells are human cells. In an exemplary embodiment, the carcinoma cells are LNCaP cells. In another exemplary embodiment, the carcinoma cells are PC3 cells.

In some preferred embodiments, the oligopeptide is produced synthetically. In exemplary embodiments, the oligopeptide is produced by solid phase synthesis and purified by high performance liquid chromatography as known in the art.

### II. Oligopeptide Formulations

The oligopeptide formulations used in the methods and medicaments of the present disclosure comprise at least one isolated oligopeptide of the preceding section, and at least one pharmaceutically acceptable excipient and/or an oral delivery agent. In some embodiments, the formulations may further comprise an enteric coating, liposomes, microspheres, or micro-/nano-particles. For instance, in some embodiments, the isolated oligopeptide is encapsulated within an enteric coating, liposomes, microspheres, or micro-/nano-particles.

The amount of an oligopeptide of the disclosure, which will be effective in the treatment of a particular disorder or condition disclosed herein depends on the nature of the disorder or condition, and can be determined by standard clinical techniques. In addition, in vitro or in vivo assays may optionally be employed to help identify optimal dosage ranges. In some embodiments, the dose of the oligopeptide of the present disclosure is from about 0.1 mg/kg to about 1000 mg/kg, about 1.0 mg/kg to about 100 mg/kg, or about 10 mg/kg body weight of the subject to be treated. In some embodiments, the dose of oligopeptide is no less than 0.1, 0.5, 1.0, 5.0, 10, 20, 30, 40, 50, 60, 70, 80, 90, 100, or 500 mg/kg, and/or the dose of the oligopeptide is no more than 1000, 500, 100, 90, 80, 70, 60, 50, 40, 30, 20, 10, 5.0, 1.0, or 0.5 mg/kg, in which the lower limit is less than the upper limit.

### A. Excipients

Pharmaceutically acceptable excipients of the present disclosure, include for instance, solvents, bulking agents, buffering agents, tonicity adjusting agents, and preservatives (Pramanick et al., Pharma Times, 45:65-77, 2013). In some embodiments the formulations may comprise an excipient that functions as one or more of a solvent, a bulking agent, a buffering agent, and a tonicity adjusting agent (e.g., sodium chloride in saline may serve as both an aqueous vehicle and a tonicity adjusting agent).

In some embodiments, the formulations comprise an aqueous vehicle as a solvent. Suitable vehicles include for instance sterile water, saline solution, phosphate buffered saline, and Ringer's solution. In some embodiments, the formulation is isotonic.

The formulations may comprise a buffering agent. Buffering agents control pH to inhibit degradation of the active agent during processing, storage and optionally reconstitution. Suitable buffers include for instance salts comprising acetate, citrate, phosphate or sulfate. Other suitable buffers include for instance amino acids such as arginine, glycine, histidine, and lysine. The buffering agent may further comprise hydrochloric acid or sodium hydroxide. In some embodiments, the buffering agent maintains the pH of the formulation within a range of 6 to 9. In some embodiments, the pH is greater than (lower limit) 6, 7 or 8. In some embodiments, the pH is less than (upper limit) 9, 8, or 7. That is, the pH is in the range of from about 6 to 9 in which the lower limit is less than the upper limit.

The formulations may comprise a tonicity adjusting agent. Suitable tonicity adjusting agents include for instance dextrose, glycerol, sodium chloride, glycerin and mannitol

The formulations may comprise a bulking agent. Bulking agents are particularly useful when the pharmaceutical formulation is to be lyophilized before administration. In some embodiments, the bulking agent is a protectant that aids in the stabilization and prevention of degradation of the active agents during freeze or spray drying and/or during storage. Suitable bulking agents are sugars (mono-, di- and polysaccharides) such as sucrose, lactose, trehalose, mannitol, sorbital, glucose and raffinose.

The formulations may comprise a preservative. Suitable preservatives include for instance antioxidants and antimicrobial agents. However, in preferred embodiments, the formulation is prepared under sterile conditions and is in a single use container, and thus does not necessitate inclusion of a preservative.

### B. Oral Delivery Agents

Oral delivery agents of the present disclosure include, for instance, absorption enhancers, fatty acids, enzyme inhibitors, polyethylene glycol, mucoadhesive polymers, and cell penetrating peptides (Dan et al., Children, 7:307, 2020).

Commonly utilized routes of administration for therapeutic peptides and proteins include intravenous (IV), intraperitoneal (IP), and intramuscular (IM) injections. However, oral administration is preferred by patients and oral medications are typically less expensive to manufacture, distribute and administer. Unfortunately, development of orally available dosage forms of therapeutic peptides and proteins have been complicated for a variety of reasons, including but not limited to poor stability in physiological conditions, short biological half-life, and low permeability through the epithelial barrier in the small intestine. Thus, in some embodiments, the formulations of the present disclosure are designed to protect the isolated oligopeptide from the proteolytic enzymes and acidic environment found in the stomach, such that their bioactivity is retained as they are absorbed into the bloodstream (see, e.g., Dan et al., Children, 7:307, 2020).

### III. Non-Steroidal Anti-Androgens

An antiandrogen is a compound that inhibits the activity of an androgen. Antiandrogens are classified as steroidal anti-androgens or non-steroidal anti-androgens (NSAAs). The methods and medicaments of the present disclosure comprise a NSAA. In some preferred embodiments, the NSAA is an androgen receptor antagonist that blocks the effects of testosterone and dihydrotestosterone. NSAAs are commonly used in combination with castration as a combined androgen blockade for treatment of prostate cancer (Crawford et al., J Urol, 200(5):956-966, 2018). In some embodiments, the NSAA is selected from the group consisting of bicalutamide, apalutamide, enzalutamide, flutamide, nilutamide, topilutamide, darolutamide, proxalutamide, and combinations thereof. In some embodiments, the nonsteroidal antiandrogen is bicalutamide.

### IV. Methods of Use

The salmon protein hydrolysates, isolated oligopeptides and formulations thereof of the present disclosure find use in combination with a nonsteroidal antiandrogen in methods and medicaments for inhibiting proliferation of a carcinoma cell. In some embodiments, the carcinoma cell is an adenocarcinoma cell. In some embodiments, the carcinoma cell is a prostate cancer cell. In some embodiments, the prostate cancer cell secretes prostate-specific antigen. In some embodiments, the prostate cancer cell is androgen receptor-positive. In some embodiments, wherein the prostate cancer cell is androgen receptor-negative. In some embodiments, the carcinoma cell is a human cell. In some preferred embodiments, the salmon protein hydrolysates, isolated oligopeptides and formulations thereof of the present disclosure find use in combination with a nonsteroidal antiandrogen in methods and medicaments for treating prostate cancer in a human subject in need thereof.

In some in vivo embodiments, the formulation is administered by mouth. For instance, the formulation may be administered enterically. In some embodiments, the formulation is administered by a buccal, a sublabial, or a sublingual route. In some embodiments, the nonsteroidal antiandrogen is administered orally.

In some embodiments, the isolated oligopeptide is capable of increasing expression of ferritin heavy chain 1 (FTH1) mRNA by carcinoma cells when contacted with the oligopeptide in the presence of the nonsteroidal antiandrogen. In some embodiments, the isolated oligopeptide is capable of increasing expression of FTH1 mRNA, wherein the increase of FTH1 mRNA is relative to carcinoma cells contacted with the nonsteroidal antiandrogen in the absence of the oligopeptide. In some embodiments, the isolated oligopeptide is capable of increasing expression of FTH1 mRNA, wherein the increase of FTH1 mRNA is relative to carcinoma cells cultured under the same condition except for the absence of the oligopeptide. In some embodiments, the isolated oligopeptide is capable of increasing expression of FTH1 mRNA, wherein the increase of FTH1 mRNA is relative to carcinoma cells cultured under the same condition except for the absence of the oligopeptide. In some embodiments, the isolated oligopeptide is capable of increasing expression of FTH1 mRNA, wherein the increase of FTH1 mRNA is relative to carcinoma cells cultured under the same condition except for the presence of a negative control oligopeptide.

In some embodiments, the isolated oligopeptide is capable of decreasing expression of transferrin receptor 1 (TFRC) mRNA by carcinoma cells when contacted with the oligopeptide in the presence of the nonsteroidal antiandrogen. In some embodiments, the isolated oligopeptide is capable of decreasing expression of TFRC mRNA, wherein the decrease of TFRC mRNA is relative to carcinoma cells contacted with the nonsteroidal antiandrogen in the absence of the oligopeptide. In some embodiments, the isolated oligopeptide is capable of decreasing expression of TFRC mRNA, wherein the decrease of TFRC mRNA is relative to carcinoma cells cultured under the same condition except for the absence of the oligopeptide. In some embodiments, the isolated oligopeptide is capable of decreasing expression of TFRC mRNA, wherein the decrease of TFRC mRNA is relative to carcinoma cells cultured under the same condition except for the absence of the oligopeptide. In some embodiments, the isolated oligopeptide is capable of decreasing expression of TFRC mRNA, wherein the decrease of TFRC mRNA is relative to carcinoma cells cultured under the same condition except for the presence of a negative control oligopeptide.

In some aspects, the present disclosure provides methods and medicaments for inhibiting proliferation of a carcinoma cell, comprising contacting the carcinoma cell with an effective amount of a nonsteroidal antiandrogen and an effective amount of an oligopeptide (or formulation comprising the oligopeptide). In some embodiments, the contacting is in vivo.

In some aspects, the present disclosure provides methods and medicaments for treating prostate cancer in a mammalian subject in need thereof, comprising administering to the subject an effective amount of a nonsteroidal antiandrogen and an effective amount of a an oligopeptide (or formulation comprising the oligopeptide). In some embodiments, cells of the prostate cancer secrete prostate-specific antigen. In some embodiments, the prostate cancer is a metastatic carcinoma. In some embodiments, wherein the prostate cancer cell is androgen receptor-positive. In some embodiments, wherein the prostate cancer cell is androgen receptor-negative.

### Enumerated Embodiments

1. A method for inhibiting proliferation of a carcinoma cell, comprising contacting the carcinoma cell with an effective amount of a nonsteroidal antiandrogen and an effective amount of a formulation comprising an oligopeptide consisting of the amino acid sequence of Xm(R/D)EES(G/D)(E/K)Xn (Consensus No. 1), in which m and n are integers independently selected from the range of from 0-10, and each X, if present, is independently selected from any amino acid.

2. A method for inhibiting proliferation of a carcinoma cell, comprising contacting the carcinoma cell with an effective amount of a nonsteroidal antiandrogen and an effective amount of a formulation comprising an oligopeptide consisting of the amino acid sequence of Xp(R/D)EESGEPXq (SEQ ID NO:10), in which p is an integer selected from the range of from 0-10, q is an integer selected from the range of from 0-9, and each X, if present, is independently selected from any amino acid.

3. The method of embodiment 2, comprising the amino acid sequence of Xr(R/D)EESGEP (SEQ ID NO:11), in which Xr is leucine or absent.

4. The method of embodiment 3, wherein the oligopeptide comprises the amino acid sequence of REESGEP (SEQ ID NO:2).

5. The method of embodiment 3, wherein the oligopeptide comprises the amino acid sequence of LDEESGEP (SEQ ID NO:5).

6. The method of embodiment 1, wherein the oligopeptide comprises the amino acid sequence of REESGE (SEQ ID NO:1), REESGEP (SEQ ID NO:2), KEEDEESGE (SEQ ID NO:3), KPREESGE (SEQ ID NO:4), LDEESGEP (SEQ ID NO:5), REESDKPMY (SEQ ID NO:6), PREESDKP (SEQ ID NO:7), or REESGEL (SEQ ID NO:8).

7. The method of any one of embodiments 1-6, wherein the oligopeptide is capable of increasing expression of ferritin heavy chain 1 (FTH1) mRNA by carcinoma cells contacted with the oligopeptide in the presence of an antiandrogen.

8. The method of embodiment 1, wherein the formulation comprises a fish protein hydrolysate, optionally wherein the fish protein hydrolysate is a salmon protein hydrolysate.

9. The method of any one of embodiments 1-8, wherein the nonsteroidal antiandrogen is selected from bicalutamide, apalutamide, enzalutamide, flutamide, nilutamide, topilutamide, darolutamide, proxalutamide, and combinations thereof.

10. The method of embodiment 9, wherein the nonsteroidal antiandrogen is bicalutamide.

11. The method of embodiment 9, wherein the nonsteroidal antiandrogen is enzalutamide.

12. The method of any one of embodiments 1-11, wherein the carcinoma cell is an adenocarcinoma cell.

13. The method of any one of embodiments 1-11, wherein the carcinoma cell is a prostate cancer cell.

14. The method of embodiment 13, wherein the prostate cancer cell is androgen receptor-positive.

15. The method of embodiment 13, wherein the prostate cancer cell is androgen receptor-negative.

16. The method of any one of embodiments 1-15, wherein the carcinoma cell is a human cell.

17. The method of any one of embodiments 1-16, wherein the contacting is in vivo.

18. The method of any one of embodiments 1-17, wherein the formulation comprising the oligopeptide further comprises at least one pharmaceutically acceptable excipient.

19. The method of any one of embodiments 1-18, wherein the formulation comprising the oligopeptide further comprises an oral delivery agent.

20. The method of embodiment 19, wherein the oral delivery agent comprises an absorption enhancer, a fatty acid, an enzyme inhibitor, polyethylene glycol, a mucoadhesive polymer, a cell penetrating peptide, or a combination thereof.

21. The method of embodiment 19 or embodiment 20, further comprising an enteric coating, liposomes, microspheres, and/or micro-/nano-particles.

22. A method for treating prostate cancer in a mammalian subject in need thereof, comprising: administering to the subject i) an effective amount of a nonsteroidal antiandrogen and ii) an effective amount of a formulation comprising an oligopeptide consisting of the amino acid sequence of Xm(R/D)EES(G/D)(E/K)Xn (Consensus No. 1), in which m and n are integers independently selected from the range of from 0-10, and each X, if present, is independently selected from any amino acid.

23. The method of embodiment 22, wherein the antiandrogen and the formulation are administered by mouth.

24. The method of embodiment 23, wherein the formulation is administered enterically.

25. The method of embodiment 24, wherein the formulation is administered by a buccal, a sublabial, or a sublingual route.

26. The method of any one of embodiments 22-25, wherein administration of the antiandrogen and the formulation result in a reduction in volume of the prostate cancer relative to the volume prior to the treatment.

27. The method of any one of embodiments 22-26, wherein the mammalian subject is a human subject.

28. The method of any one of embodiments 22-27, wherein the oligopeptide comprises the amino acid sequence of REESGEP (SEQ ID NO:2).

29. The method of any one of embodiments 22-27, wherein the oligopeptide comprises the amino acid sequence of LDEESGEP (SEQ ID NO:5).

30. The method of any one of embodiments 22-27, wherein the oligopeptide comprises the amino acid sequence of REESGE (SEQ ID NO:1), REESGEP (SEQ ID NO:2), KEEDEESGE (SEQ ID NO:3), KPREESGE (SEQ ID NO:4), LDEESGEP (SEQ ID NO:5), REESDKPMY (SEQ ID NO:6), PREESDKP (SEQ ID NO:7), or REESGEL (SEQ ID NO:8).

31. The method of any one of embodiments 22-27, wherein the formulation comprises a fish protein hydrolysate, optionally wherein the fish protein hydrolysate is a salmon protein hydrolysate.

32. The method of any one of embodiments 22-31, wherein the nonsteroidal antiandrogen is selected from bicalutamide, apalutamide, enzalutamide, flutamide, nilutamide, topilutamide, darolutamide, proxalutamide, and combinations thereof.

33. The method of embodiment 32, wherein the nonsteroidal antiandrogen is bicalutamide.

34. The method of embodiment 32, wherein the nonsteroidal antiandrogen is enzalutamide.

35. The method of any one of embodiments 22-34, further comprising administering a gonadotropin-releasing hormone antagonist.

36. The method of any one of embodiments 22-35, wherein the prostate cancer is androgen receptor-positive.

37. The method of any one of embodiments 22-35, wherein the prostate cancer is androgen receptor-negative.

38. The method of any one of embodiments 22-37, wherein the prostate cancer is a metastatic carcinoma.

### EXAMPLES

Abbreviations: ACTB (beta-actin); AR (androgen receptor); BIC (bicalutamide), ENZ (enzalutamide); FTH1 (ferritin heavy chain 1); MIC (minimum inhibitory concentration); SPH (salmon protein hydrolysate), TFRC or TFR1 (transferrin receptor 1); −ve (negative); +ve (positive).

### Example 1

### Preparation of Biologically Active Salmon Protein Hydrolysate

Salmon Protein Hydrolysate (SPH) powder was produced by enzymatic hydrolysis of salmon (Salmo salar) head and backbone post filleting as described (US 2021/0252099). Briefly, 1000 grams of ground head and backbone was added to 1000 ml of water and the mixture was heated to 50° C. 10 g of an endopeptidase enzyme (pepsin) was added and the mixture was stirred for 30 minutes. Then 10 g of an exopeptidase enzyme (carboxypeptidase) was added and the mixture was stirred for 15 minutes. Next 5 grams of Flavourzyme® (a blend of endo- and exo-proteases derived from Aspergillus oryzae, marketed by Novozymes A/S, Bagsvaerd, Denmark) was added and the mixture was stirred for 10 minutes. The endopeptidase and exopeptidase treated salmon protein mixture was subsequently heated to 85° C. for 15 minutes to inactivate the proteases. After filtering, the hydrolysate fraction was concentrated to 30% dry matter in a conventional evaporator and spray-dried to yield salmon protein hydrolysate powder.

### Example 2

### Determination of Effects of Salmon Protein Hydrolysate in Combination with an Antiandrogen on Androgen Receptor Positive and Negative Cell Lines

This example describes the effects of salmon protein hydrolysate (SPH) and the antiandrogen, bicalutamide, on proliferation and gene expression of two prostate cancer cell lines in vitro.

### Materials and Methods

Test Solutions. SPH test solutions were prepared by adding 10 μg, 40 μg, or 160 μg of SPH powder to 1 ml of DMEM containing 0.3% FBS+2% DMSO and sonicating for 10 min just prior to use. Bicalutamide, referred to herein as BIC, (Sigma-Aldrich, Catalog No. B9061) test solutions were prepared by adding 0.43 or 4.3 μg to 1 ml (1 μM and 10 μM) of DMEM containing 0.3% FBS+2% DMSO and sonicating for 10 min just prior to use. Enzalutamide, referred to herein as ENZ (Intas Pharmaceuticals, India), test solutions were prepared by serial dilution of a 1 mM stock solution (3% DMS in water) into culture medium.

Cell Culture. The C4 subline (CRL-3313™, RRID: CVCL 4783), which was derived from the human prostate carcinoma cell line LNCaP, was obtained from ATCC (Manassas, VA). C4, which is referred to herein as LNCaP, is an exemplary androgen receptor-positive (AR+ve) cell line that has an epithelial-like morphology and a low metastatic potential. The PC-3 cell line (CRL-1435, RRID: CVCL_0035) was obtained from ATCC (Manassas, VA). PC-3 is an exemplary androgen receptor-negative (AR−ve) cell line that has an epithelial morphology and a high metastatic potential. The Vertebral-Cancer of the Prostate (VCaP) cell line was obtained from Sigma-Aldrich, USA (Catalog No. 06020201). VCaP is a human cell line with an epithelial morphology that was established from a metastatic prostate cancer lesion (Korenchuk et al., In Vivo, 15(2):163˜168, 2001). VCaP cells are androgen receptor-positive and express high levels of prostate-specific antigen. The VCaP-EnzR cell line was also obtained from Sigma-Aldrich, USA (Catalog No. SCC421). VCaP-EnzR is a human cell line, which was derived from VCaP after prolonged culture in the presence of enzalutamide, an androgen receptor antagonist (Kregel et al., Oncotarget, 7(18):26529-26274, 2016). VCaP-EnzR cells are enzalutamide-resistant and therefore are a suitable model for studying castration-resistant prostate cancer. All cell lines were cultured in RPMI1640 supplemented with 2 mmol/L L-glutamine. 100 U/mL penicillin, 100 mg/mL streptomycin, and 10% heat-inactivated fetal bovine serum. All cells were certified negative for Mycoplasma.

Clonogenic Assay. Confluent cells were trypsinized and seeded at densities ranging from 3000 to 5000 cells in 10 cm Petri dishes, depending on the cell line, as recommended in the ATCC or Sigma-Aldrich product sheets. Cells were allowed to adhere for 24-48 hours before application of treatment with either SPH alone (10-160 μg/ml), bicalutamide (LNCaP and PC3 cells) alone or enzalutamide (VCaP or VCaP-EnzR. cells) alone, or a combination of SPH and BIC or ENZ. In the combination treatment, the doses of bicalutamide and enzalutamide were fixed by defining the minimum inhibitory concentrations (MIC) to begin inhibiting colony formation. Treatments were applied daily for 5 days without media changes. The colonies were allowed to develop over 12 days, stained with crystal violet and automatically counted using a TC20 Automated Cell Counter (Bio Rad, Hercules, CA). Each assay was internally controlled using untreated cells (cell media+0.1% DMSO). Relative plating efficiencies were expressed as percentages relative to the plating efficiency of untreated cells and reported as the relative percent colony survival value. All experiments were performed in triplicate.

Gene Expression. LNCaP, PC-3, VCaP, and VCaP-EnzR cells were seeded in 24-well plates and maintained in RPMI1640 medium supplemented with 2 mmol/L L-glutamine. 100 U/mL penicillin, 100 mg/mL streptomycin, and 10% heat-inactivated fetal bovine serum at 37° C. in a humidified 5% CO₂ atmosphere. 24-48 hours later the cells were incubated with the SPH and bicalutamide (BIC) or enzalutamide (ENZ) concentrations that showed significant decreases in relative percent colony survival rates in both cell lines:
LNCaP cells: SPH at 40 and 160 μg/ml together with 1 μM BIC;
PC3 cells: SPH at 40 and 160 μg/ml together with 10 μM BIC;
VCaP cells: SPH at 160 μg/ml together with 1.4 μM ENZ; and
VCaP-EnzR cells: SPH at 160 μg/ml together with 47 μM ENZ.
   SPH absent wells (DMSO 0.04%) were used as the negative control. All experiments were run in triplicate.

Samples were studied for expression of the ferritin heavy chain 1 (FTH1) and transferrin receptor 1 (TERC) genes. Total RNA was extracted from cell pellets using the UPzol reagent (Biotechrabbit), followed by DNAse treatment (DNAse TURBO) according to the manufacturers' protocols. Complementary DNA (cDNA) was synthesized with the High Capacity cDNA Reverse Transcription Kit (Applied Biosystems) using random hexamers. Gene expression levels were measured by qRT-PCR.

1 μl of cDNA (corresponding to 50 ng of reverse transcribed RNA) was amplified by Qpcr (QuantStudio™ 6 Flex Real-Time PCR System), using TaqMan™ Universal PCR Master Mix (Catalog No. 4304437) and a TaqMan™ assay (Roche Molecular Systems, Inc., Pleasanton, CA). FTH1 and TFRC gene expression was estimated relative to expression of the housekeeping gene, beta-actin (ACTB), following the standard formula: 2-ΔCt. TaqMan probe IDs used include: (i) FTH1, Hs01694011_s1; (ii) TFRC, Hs00951083_m1; and (iii) ACTB, Hs01060665_g1.

Statistical Analyses. Statistical differences were determined by paired, two-tailed Student t-test between treatment groups in each cell line.

### Results

Cell Proliferation. Combining SPH with bicalutamide significantly reduced colony formation rates in AR+ve and AR−ve prostate cancer cell lines as shown in FIG. 1 (LNCaP AR+ve cells) and FIG. 2 (PC3 AR−ve cells), respectively. In contrast, exposure to 10-160 μg/ml SPH alone did not result in significant dose-dependent reduction in colony formation in either LNCaP or PC3 cells. Similarly, combining SPH with enzalutamide reduced colony formation rates in VCaP cells as shown in FIG. 4 and VCap-EnzR cells as shown in FIG. 5, albeit at higher enzalutamide concentrations in VCap-EnzR cultures. In contrast, exposure to 40-160 μg/ml SPH alone did not result in a substantial reduction in colony formation rates in VCaP and VCaP-EnzR cells.

In the AR+ve LNCaP cell line, exposure to bicalutamide caused a significant reduction in the colony formation rate at 1 μM concentration (P<0.001) but not at 0.5 μM concentration (FIG. 1). Combining 40 and 160 μg/ml SPH with the MIC dose of 0.5 μM and the effective dose of 1.0 μM bicalutamide resulted in significant reductions in colony formation. Significant attenuation of the MIC bicalutamide activity was seen with both 40 and 160 μg/ml SPH co-treatment. 40 μg/ml SPH combined with 0.5 μM bicalutamide decreased colony survival from 85% to 68% with a further decrease to 19% colony survival when the SPH dose was increased from 40 to 160 μg/ml (P<0.001). 40 μg/ml SPH combined with 1 μM bicalutamide decreased colony survival from 25% to 4%, with a further decrease to 2% colony survival when the SPH dose was increased from 40 to 160 μg/ml (P<0.001).

In the AR−ve PC3 cell line, treatment with 10 μM bicalutamide caused a significant reduction in colony formation rate to 52% (P<0.001), whereas treatment with 1 μM bicalutamide did not result in a significant decrease in percent colony survival (FIG. 2). Combining 40 and 160 μg/ml SPH with the non-effective 1 μM bicalutamide dose did not result in reductions in percent colony formation rates in PC3 AR−ve cells. However, combining 40 and 160 μg/ml SPH with 10 μM bicalutamide did result in significant reductions in percent colony formation rates in PC3 cells. 40 μg/ml SPH combined with 10 μM bicalutamide decreased colony survival from 52% to 41%, with a further decrease to 32% colony survival when the SPH dose was increased from 40 to 160 μg/ml (P<0.01).

Gene Expression. Two iron homeostasis genes, FTH1 and TFRC, were examined for changes in expression in LNCaP AR+ve and PC3 AR−ve cells when treated with bicalutamide alone or with bicalutamide in combination with SPH. In both cell lines, SPH co-treatment with bicalutamide led to changes in gene expression with FTH1 mRNA expression upregulated by greater than two-fold and concomitant TFRC mRNA expression downregulated by greater than two-fold.

FTH1 and TFRC gene expression levels were also measured in VCaP and VcaP-ENZR cells when treated with enzalutamide alone or with enzalutamide in combination with SPH. In both cell lines, SPH co-treatment with enzalutamide led to increased expression of FTH1 mRNA and decreased expression of TFRC.

Although numerical differences between cell lines (PC3 cells showed greater changes in gene expression) and [SPH] dose response was noted, no statistically significant conclusions were drawn.

TABLE 2-1
Effects of SPH and Bicalutamide on Gene Expression

| Cell Line | Treatment | Symbol | Fold Change | | | Average | STD |
|---|---|---|---|---|---|---|---|
| LNCaP | 1 μM BIC | FTH1 | 1.0 | 1.1 | 1.0 | 1.0 | 0.1 |
| LNCaP | 1 μM BIC | TFRC | 1.0 | 1.0 | 1.2 | 1.1 | 0.1 |
| LNCaP | 40 SPH + 1 BIC | FTH1 | 2.3 | 2.2 | 2.3 | 2.3 | 0.1 |
| LNCaP | 40 SPH + 1 BIC | TFRC | 0.5 | 0.6 | 0.5 | 0.5 | 0.1 |
| LNCaP | 160 SPH + 1 BIC | FTH1 | 2.8 | 3.0 | 2.7 | 2.8 | 0.2 |
| LNCaP | 160 SPH + 1 BIC | TFRC | 0.3 | 0.4 | 0.5 | 0.4 | 0.1 |
| PC3 | 10 μM BIC | FTH1 | 0.9 | 1.0 | 1.2 | 1.0 | 0.2 |
| PC3 | 10 μM BIC | TFRC | 1.0 | 1.1 | 1.2 | 1.1 | 0.1 |
| PC3 | 40 SPH + 10 BIC | FTH1 | 2.2 | 2.4 | 2.5 | 2.4 | 0.2 |
| PC3 | 40 SPH + 10 BIC | TFRC | 0.5 | 0.5 | 0.4 | 0.5 | 0.1 |
| PC3 | 160 SPH + 10 BIC | FTH1 | 2.8 | 2.6 | 2.4 | 2.6 | 0.2 |
| PC3 | 160 SPH + 10 BIC | TFRC | 0.3 | 0.3 | 0.4 | 0.3 | 0.1 |

TABLE 2-2
Effects of SPH and Enzalutamide on Gene Expression

| Cell Line | Treatment (IC50) | Symbol | Fold Change | | | Average | STD |
|---|---|---|---|---|---|---|---|
| VCaP | 1.4 μM ENZ | FTH1 | 1.1 | 1.1 | 1.0 | 1.07 | 0.06 |
| VCaP | 1.4 μM ENZ | TFRC | 1.1 | 1.0 | 1.0 | 1.03 | 0.06 |
| VCaP | 160 SPH + 1.4 ENZ | FTH1 | 1.9 | 2.3 | 1.8 | 2.00 | 0.26 |
| VCaP | 160 SPH + 1.4 ENZ | TFRC | 0.6 | 0.8 | 0.8 | 0.73 | 0.12 |
| VCaP-ENZR | 47 μM ENZ | FTH1 | 0.7 | 0.8 | 0.8 | 0.77 | 0.06 |
| VCaP-ENZR | 47 μM ENZ | TFRC | 1.1 | 1.0 | 1.3 | 1.13 | 0.15 |
| VCaP-ENZR | 160 SPH + 47 ENZ | FTH1 | 2.5 | 2.7 | 2.9 | 2.70 | 0.20 |
| VCaP-ENZR | 160 SPH + 47 ENZ | TFRC | 0.5 | 0.6 | 0.6 | 0.57 | 0.06 |

### Example 3

### Effects of Oligopeptides in Combination with an Antiandrogen on Human Prostate Cancer Cell Lines

This example describes the effects of oligopeptides and an antiandrogen (bicalutamide or enzalutamide), on proliferation and gene expression of human prostate cancer cell lines in vitro.

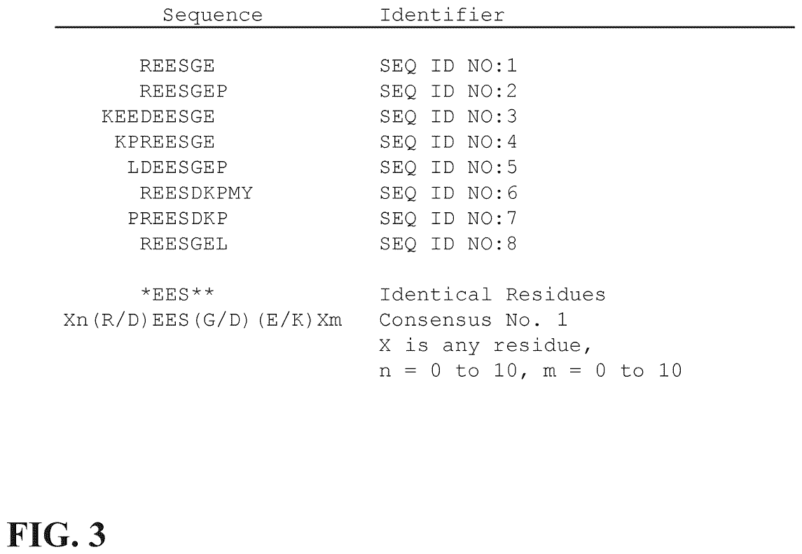

The four human prostate cancer cell lines utilized in this example, as well as the experimental design are as described in Example 2. The oligopeptides tested were produced by solid phase synthesis and obtained from Biomatik, Canada. The amino acid sequences of the oligopeptides are shown below in Table 3-1, and an alignment of their sequences and a consensus are shown in FIG. 3.

TABLE 3-1
Oligopeptide Numbers, Sequences and Identifiers

| Oligopeptide | | |
|---|---|---|
| No. | Sequence | Identifier |
| FT-001 | REESGE | SEQ ID NO: 1 |
| FT-002 | REESGEP | SEQ ID NO: 2 |
| FT-008 | REESGEL | SEQ ID NO: 8 |
| FT-004 | KPREESGE | SEQ ID NO: 4 |
| FT-005 | LDEESGEP | SEQ ID NO: 5 |
| FT-003 | KEEDEESGE | SEQ ID NO: 3 |
| FT-006 | REESDKPMY | SEQ ID NO: 6 |
| FT-007 | PREESDKP | SEQ ID NO: 7 |

All oligopeptides were tested in combination with BIC or ENZ in clonogenic assays. Effects on gene expression of the more active oligopeptides from the clonogenic assays were also tested. The treatment conditions for each cell line included:
LNCaP cells: (i) oligopeptides at 10 μM with 0.4 μM BIC, (ii) 0.4 μM BIC alone, or (iii) SPH at 160 μg/ml with 0.4 μM BIC;
PC3 cells: (i) oligopeptides at 10 μM with 10 μM BIC, (ii) 10 μM BIC alone, or (iii) SPH at 160 μg/ml with 10 μM BIC;
VCaP cells: (i) oligopeptides at 10 μM with 1.4 μM ENZ, (ii) 1.4 μM ENZ alone, or (iii) SPH at 160 μg/ml with 1.4 μM ENZ; and
VCaP-EnzR cells: (i) oligopeptides at 10 μM with 47 μM ENZ, (ii) 47 μM, ENZ alone, or (iii) SPH at 160 μg/ml with 47 μM ENZ.
   SPH absent wells (DMSO 0.04%) were used as the negative control. All experiments were run in triplicate.

### Results

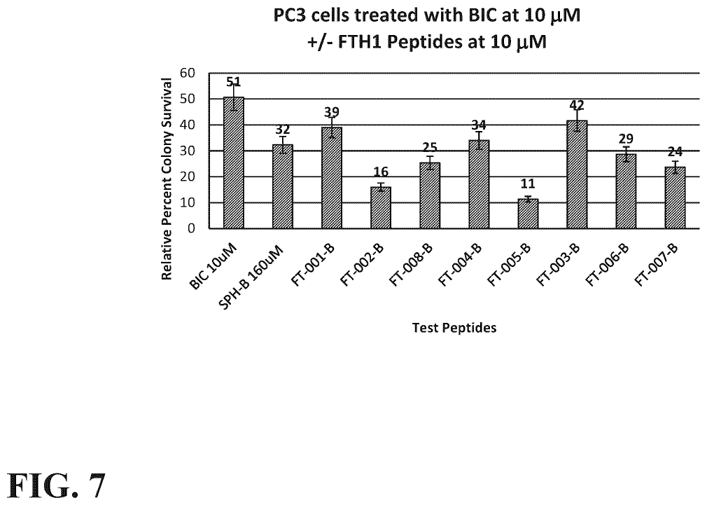

Cell survival. When 10 μM FT-002 or FT-005 oligopeptide were co-administered with 0.4 μM bicalutamide (IC50), LNCaP cells were found to display significantly decreased survival by >70% compared to 0.4 μM bicalutamide (IC50) monotreatment (p<0.001), as shown in FIG. 6. Similarly, PC3 cells displayed an approximately 70% decrease in survival compared to 10 μM bicalutamide (IC50) monotreatment, and >50% (p<0.001) decrease in survival compared to the combination of bicalutamide with 160 μM SPH, as shown in FIG. 7. FT-002 or FT-005 in conjunction with bicalutamide also diminished colony survival >50% compared to the combination of bicalutamide with 160 μM SPH

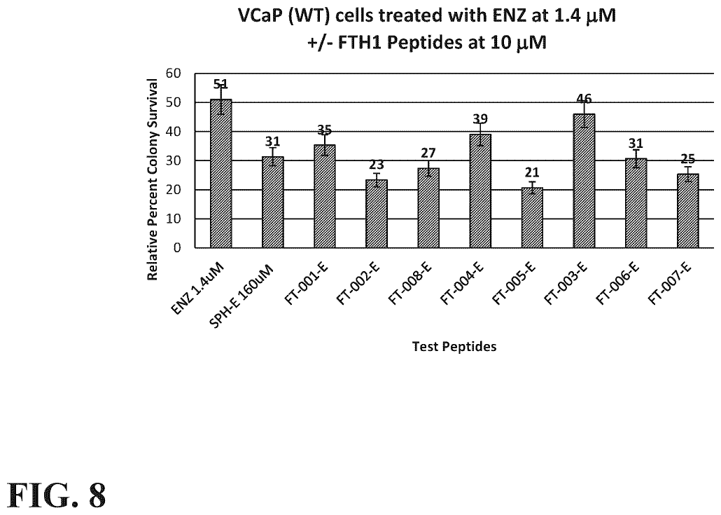

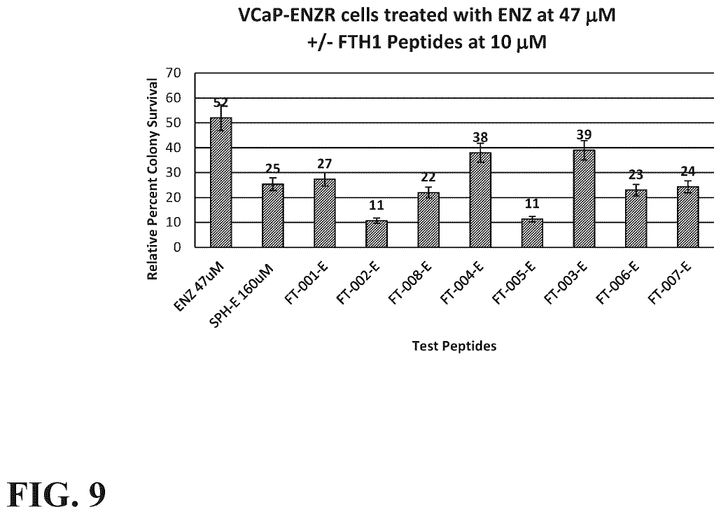

VCaP cells treated with 10 μM FT-002 or FT-005 oligopeptide in combination with 1.4 μM enzalutamide (IC50) showed a significant, >50% decrease in cell survival compared to 1.4 μM enzalutamide alone (p<0.001), and an approximately 40% decrease (p<0.01) compared to the combination of 1.4 μM enzalutamide and 160 μM SPH treatment, as shown in FIG. 8. Relative survival of VCaP-EnzR cells was significantly diminished from 52% (47 μM enzalutamide monotreatment) down to 11% when treated concomitantly with 10 μM FT-002 or FT-005 oligopeptide and 47 μM enzalutamide (p<0.001), as shown in FIG. 9. FT-002 or FT-005 oligopeptide in combination with enzalutamide also significantly diminished colony survival as compared to treatment with enzalutamide in combination with 160 μM SPH treatment (>50%, p<0.001).

Gene Expression. Two iron homeostasis genes, FTH1 and TFRC, were examined for changes in expression in LNCaP, PC3, VCaP, and VCaP-EnzR cells treated with bicalutamide or enzalutamide in the presence of an oligopeptide. Differential gene expression analysis revealed upregulation by greater than twofold of FTH1 gene expression and a consistent downregulation of the transferrin receptor gene across all cell lines.

TABLE 3-2
Effects of Oligopeptides and Bicalutamide on Gene Expression

| Cell Line | Peptide Treatment | Gene Symbol | Fold Change | | | Average | STD |
|---|---|---|---|---|---|---|---|
| LNCaP | FT-002-B | FTH1 | 2.1 | 1.9 | 2.1 | 2.0 | 0.1 |
| LNCaP | FT-002-B | TFRC | 0.7 | 0.7 | 0.7 | 0.7 | 0.0 |
| LNCaP | FT-005-B | FTH1 | 2.2 | 2.2 | 2.3 | 2.2 | 0.1 |
| LNCaP | FT-005-B | TFRC | 0.7 | 0.6 | 0.6 | 0.6 | 0.1 |
| LNCaP | FT-008-B | FTH1 | 1.8 | 1.9 | 2.2 | 2.0 | 0.2 |
| LNCaP | FT-008-B | TFRC | 0.8 | 0.9 | 0.8 | 0.8 | 0.1 |
| LNCaP | FT-006-B | FTH1 | 1.9 | 1.9 | 1.6 | 1.8 | 0.2 |
| LNCaP | FT-006-B | TFRC | 0.9 | 0.9 | 0.8 | 0.9 | 0.1 |
| PC3 | FT-002-B | FTH1 | 2.0 | 2.1 | 2.0 | 2.0 | 0.1 |
| PC3 | FT-002-B | TFRC | 0.7 | 0.8 | 0.6 | 0.7 | 0.1 |
| PC3 | FT-005-B | FTH1 | 2.2 | 2.1 | 2.1 | 2.1 | 0.1 |
| PC3 | FT-005-B | TFRC | 0.7 | 0.5 | 0.5 | 0.6 | 0.1 |
| PC3 | FT-007-B | FTH1 | 1.9 | 1.9 | 2.0 | 1.9 | 0.1 |
| PC3 | FT-007-B | TFRC | 0.6 | 0.8 | 0.8 | 0.7 | 0.1 |
| PC3 | FT-008-B | FTH1 | 1.8 | 1.8 | 1.9 | 1.8 | 0.1 |
| PC3 | FT-008-B | TFRC | 0.8 | 0.9 | 0.9 | 0.9 | 0.1 |

TABLE 3-3
Effects of Oligopeptides and Enzalutamide on Gene Expression

| | Peptide | Gene | | | | | |
|---|---|---|---|---|---|---|---|
| Cell Line | Treatment | Symbol | Fold Change | | | Average | STD |
| VCaP | FT-002-E | FTH1 | 2.3 | 2.1 | 2.1 | 2.2 | 0.1 |
| VCaP | FT-002-E | TFRC | 0.7 | 0.6 | 0.6 | 0.6 | 0.1 |
| VCaP | FT-005-E | FTH1 | 2.2 | 2.4 | 2.4 | 2.3 | 0.1 |
| VCaP | FT-005-E | TFRC | 0.6 | 0.6 | 0.7 | 0.6 | 0.1 |
| VCaP | FT-008-E | FTH1 | 2.1 | 2.3 | 2.1 | 2.2 | 0.1 |
| VCaP | FT-008-E | TFRC | 0.7 | 0.7 | 0.7 | 0.7 | 0.0 |
| VCaP | FT-007-E | FTH1 | 2.0 | 1.9 | 1.9 | 1.9 | 0.1 |
| VCaP | FT-007-E | TFRC | 0.8 | 0.7 | 0.8 | 0.8 | 0.1 |
| VCaP-ENZR | FT-002-E | FTH1 | 3.9 | 3.3 | 3.5 | 3.6 | 0.3 |
| VCaP-ENZR | FT-002-B | TFRC | 0.6 | 0.5 | 0.5 | 0.5 | 0.1 |
| VCaP-ENZR | FT-005-E | FTH1 | 3.1 | 3.3 | 3.2 | 3.2 | 0.1 |
| VCaP-ENZR | FT-005-E | TFRC | 0.4 | 0.5 | 0.5 | 0.5 | 0.1 |
| VCaP-ENZR | FT-008-E | FTH1 | 2.5 | 2.7 | 2.4 | 2.5 | 0.2 |
| VCaP-ENZR | FT-008-B | TFRC | 0.5 | 0.6 | 0.6 | 0.6 | 0.1 |
| VCaP-ENZR | FT-006-B | FTH1 | 2.4 | 2.7 | 2.8 | 2.6 | 0.2 |
| VCaP-ENZR | FT-006-B | TFRC | 0.6 | 0.7 | 0.6 | 0.6 | 0.1 |

### Example 4

### Assessment of Oligopeptides in Combination with an Antiandrogen on Human Prostate Cancer Xenografts

This example describes the effects of salmon protein hydrolysate (SPH) or oligopeptides in combination with an antiandrogen (bicalutamide or enzalutamide) on tumor growth in vivo.

LNCaP xenograft and PC3 xenograft mouse models are used to study the efficacy of an oligopeptide and an antiandrogen to treat prostate cancer. Mice used in this study are male NSG™ (NSG™; NOD.Cg-Prkdc^{scid} Il2rg^{tmlWjl}/SzJ, Jackson Laboratory, Catalog No. 005557) mice (6-8-weeks-old), or other immunodeficient mouse strain. In brief, from 1.0×10⁶, 2.0×10⁶, or 5.0×10⁶ LNCaP or PC3 cells in 100 μl are administered to mice via subcutaneous injection into one flank Day 0. Mice are weighed daily and monitored for overall health. Tumor growth over time is monitored using digital calipers, and animals are scored for body condition and activity levels several times a week. When the average tumor volume reaches about 150-250 mm³, mice are randomized into study groups (6-10 mice each). The study groups may include some or all of: (i) SPH alone, (ii) bicalutamide alone, (iii) FT-002 alone, (iv) FT-005 alone, (v) bicalutamide and FT-002, and (vi) bicalutamide and FT-005. Bicalutamide is dosed orally at 15 mg/kg once per day. Oligopeptides are initially dosed by intraperitoneal injection and subsequently dosed by intraperitoneal injection or orally at a dose of 1 mg/kg, 10 mg/kg or 100 mg/kg once per day. Treatments continue for up to 3-6 weeks. However, when mice present with a tumor >2500 mm², a tumor that is ulcerated, or with a wellness score <3, they are humanely euthanized. On D24 or at the conclusion of the study, all remaining animals are euthanized. Blood may be collected prior to tumor implantation and then weekly until euthanasia to test for serum PSA levels.

A. VCaP xenograft and VCaP-EnzR. xenograft rat models are used to study the efficacy of an oligopeptide and an antiandrogen to treat prostate cancer. Due to difficult and variable growth in immunocompromised mouse models, the VCaP xenograft and VCaP-EnzR xenografts models utilize OncoRat animals (Hera BioLabs). Rats are inoculated with VCaP cells or VCaP-EnzR. cells via subcutaneous injection into one flank on Day 0. Rats are weighed daily and monitored for overall health. Tumor growth over time is monitored using digital calipers, and animals are scored for body condition and activity levels several times a week. Treatment is initiated about three weeks post-inoculation. The study groups may include some or all of: (i) SPH alone, (ii) enzalutamide alone, (iii) FT-002 alone, (iv) FT-005 alone, (v) enzalutamide and FT-002, and (vi) enzalutamide and FT-005. Enzalutamide is dosed orally at once per day. Oligopeptides are initially dosed by intraperitoneal injection and subsequently dosed by intraperitoneal injection or orally at a dose of 1 mg/kg, 10 mg/kg or 100 mg/kg once per day. Treatments continue for up to 3-6 weeks. Blood may be collected prior to tumor implantation and then weekly until euthanasia to test for serum PSA levels. e.

Although the present disclosure has been described in some detail by way of illustration and example for purposes of clarity of understanding, it will be apparent to those skilled in the art that certain changes and modifications may be practiced in light of the above teaching. Therefore, the preceding examples should not be construed as limiting the scope of the present disclosure, which is delineated by the appended claims.

1. A method for inhibiting proliferation of a carcinoma cell, comprising contacting the carcinoma cell with an effective amount of a nonsteroidal antiandrogen and an effective amount of a formulation comprising an oligopeptide consisting of the amino acid sequence of Xm(R/D)EES(G/D)(E/K)Xn (Consensus No. 1), in which m and n are integers independently selected from the range of from 0-10, and each X, if present, is independently selected from any amino acid.

2. A method for inhibiting proliferation of a carcinoma cell, comprising contacting the carcinoma cell with an effective amount of a nonsteroidal antiandrogen and an effective amount of a formulation comprising an oligopeptide consisting of the amino acid sequence of Xp(R/D)EESGEPXq (SEQ ID NO:10), in which p is an integer selected from the range of from 0-10, q is an integer selected from the range of from 0-9, and each X, if present, is independently selected from any amino acid.

3. The method of claim 2, comprising the amino acid sequence of Xr(R/D)EESGEP (SEQ ID NO:11), in which Xr is leucine or absent.

4. The method of claim 3, wherein the oligopeptide comprises the amino acid sequence of REESGEP (SEQ ID NO:2).

5. The method of claim 3, wherein the oligopeptide comprises the amino acid sequence of LDEESGEP (SEQ ID NO:5).

6. The method of claim 1, wherein the oligopeptide comprises the amino acid sequence of REESGEP (SEQ ID NO:2), LDEESGEP (SEQ ID NO:5), REESGE (SEQ ID NO:1), KEEDEESGE (SEQ ID NO:3), KPREESGE (SEQ ID NO:4), REESDKPMY (SEQ ID NO:6), PREESDKP (SEQ ID NO:7), or REESGEL (SEQ ID NO:8).

7. The method of claim 6, wherein the oligopeptide is capable of increasing expression of ferritin heavy chain 1 (FTH1) mRNA by carcinoma cells contacted with the oligopeptide in the presence of an antiandrogen.

8. The method of claim 1, wherein the formulation comprises a fish protein hydrolysate.

9. The method of claim 6, wherein the nonsteroidal antiandrogen is selected from bicalutamide, apalutamide, enzalutamide, flutamide, nilutamide, topilutamide, darolutamide, proxalutamide, and combinations thereof.

10. The method of claim 9, wherein the nonsteroidal antiandrogen is bicalutamide.

11. The method of claim 9, wherein the nonsteroidal antiandrogen is enzalutamide.

12. The method of claim 9, wherein the carcinoma cell is an adenocarcinoma cell.

13. The method of claim 9, wherein the carcinoma cell is a prostate cancer cell.

14. The method of claim 13, wherein the prostate cancer cell is androgen receptor-positive.

15. The method of claim 13, wherein the prostate cancer cell is androgen receptor-negative.

16. The method of claim 13, wherein the carcinoma cell is a human cell.

17. The method of claim 13, wherein the contacting is in vivo.

18. The method of claim 1, wherein the formulation comprising the oligopeptide further comprises at least one pharmaceutically acceptable excipient.

19. The method of claim 1, wherein the formulation comprising the oligopeptide further comprises an oral delivery agent.

20. The method of claim 19, wherein the oral delivery agent comprises an absorption enhancer, a fatty acid, an enzyme inhibitor, polyethylene glycol, a mucoadhesive polymer, a cell penetrating peptide, or a combination thereof.

21. The method of claim 20, further comprising an enteric coating, liposomes, microspheres, and/or micro-/nano-particles.

22. A method for treating prostate cancer in a mammalian subject in need thereof, comprising: administering to the subject i) an effective amount of a nonsteroidal antiandrogen and ii) an effective amount of a formulation comprising an oligopeptide consisting of the amino acid sequence of Xm(R/D)EES(G/D)(E/K)Xn (Consensus No. 1), in which m and n are integers independently selected from the range of from 0-10, and each X, if present, is independently selected from any amino acid.

23. The method of claim 22, wherein the antiandrogen and the formulation are administered by mouth.

24. The method of claim 23, wherein the formulation is administered enterically.

25. The method of claim 24, wherein the formulation is administered by a buccal, a sublabial, or a sublingual route.

26. The method of claim 22, wherein administration of the antiandrogen and the formulation result in a reduction in volume of the prostate cancer relative to the volume prior to the treatment.

27. The method of claim 22, wherein the mammalian subject is a human subject.

28. The method of claim 22, wherein the oligopeptide comprises the amino acid sequence of REESGEP (SEQ ID NO:2).

29. The method of claim 22, wherein the oligopeptide comprises the amino acid sequence of LDEESGEP (SEQ ID NO:5).

30. The method of claim 22, wherein the oligopeptide comprises the amino acid sequence of REESGE (SEQ ID NO:1), REESGEP (SEQ ID NO:2), KEEDEESGE (SEQ ID NO:3), KPREESGE (SEQ ID NO:4), LDEESGEP (SEQ ID NO:5), REESDKPMY (SEQ ID NO:6), PREESDKP (SEQ ID NO:7), or REESGEL (SEQ ID NO:8).

31. The method of claim 22, wherein the formulation comprises a fish protein hydrolysate.

32. The method of claim 22, wherein the nonsteroidal antiandrogen is selected from bicalutamide, apalutamide, enzalutamide, flutamide, nilutamide, topilutamide, darolutamide, proxalutamide, and combinations thereof.

33. The method of claim 32, wherein the nonsteroidal antiandrogen is bicalutamide.

34. The method of claim 32, wherein the nonsteroidal antiandrogen is enzalutamide.

35. The method of claim 32, further comprising administering a gonadotropin-releasing hormone antagonist.

36. The method of claim 32, wherein the prostate cancer is androgen receptor-positive.

37. The method of claim 32, wherein the prostate cancer is androgen receptor-negative.

38. The method of claim 32, wherein the prostate cancer is a metastatic carcinoma.
